(19) United States
(12) Patent Application Publication
(10) Pub. No.: US 2009/0270665 A1
(43) Pub. Date: Oct. 29, 2009

(54) DEVICE TO SEPARATE OLEFINS FROM PARAFFINS AND TO PURIFY OLEFINS AND USE THEREOF

(75) Inventors: Adelio Miguel MAGALHAES MENDES, Porto (PT); Joao Carlos Godinho De Faria Dos Santos, Porto (PT); Antonio Pedro Grilo Taveira, Porto (PT)

Correspondence Address: SUGHRUE MION, PLLC, 2100 PENNSYLVANIA AVENUE, N.W., SUITE 800, WASHINGTON, DC, 20037, US

(73) Assignee: UNIVERSIDADE DO PORTO, Porto (PT)

(21) Appl. No.: 12/237,072

(22) Filed: Sep. 24, 2008

Related U.S. Application Data
(63) Continuation of application No. PCT/PT2007/000015, filed on Mar. 26, 2007.

(30) Foreign Application Priority Data
Mar. 24, 2006 (PT) 103453

(51) Int. Cl. C07C 7/144; B01J 35/02

(52) U.S. Cl. 585/259; 422/222; 585/818

(57) ABSTRACT

The present invention discloses a device, which uses membranes, capable of separating olefins from paraffins. The device (1) considers an ultramicroporous ceramic membrane module, zeolite or silicate based, containing a fixed carrier of copper I or silver ions (#-complexant ions) inserted by ion exchange, or as a monolayer of CuCl, AgNO₃, Cu or Ag⁺ (2). Olefins have higher diffusivity and affinity to the membrane than the remaining species, therefore the bicomponent permeation selectivity becomes reinforced when compared to the ideal permeation selectivity. The purification of olefins by removal of dienes and/or alkynes, is accomplished with a zeolite membrane functionalized with Ag⁺ and having a specific catalyst in the permeate side (4), e.g. palladium nanoparticulated, for catalyzing the hydrogenation of the permeating dienes and alkynes to the corresponding olefins, thus increasing the selectivity and the driving force of the separation.

### TECHNICAL FIELD

The present invention refers to a device and process for separating olefins from paraffins using inorganic microporous membranes, and the purification of olefins by removing the dienes and alkynes. This way, the present invention fits in the technical domain of separation units and separation units with reaction. It has particular interest for the following applications:
propylene separation from propane,
ethylene separation from ethane and,
purification of propylene contaminated with small concentrations of propyne and/or allene.

### SUMMARY OF THE INVENTION

The present invention consists on a device using microporous inorganic membranes, catalytic or non-catalytic, for the separation of olefins from paraffins and/or in the purification of olefins. The device (1) considers a module of microporous ceramic membranes, zeolite or silicate based, with a fixed carrier of: copper (I), silver (π-complexant), monolayered carrier or by ionic exchange, of CuCl, AgNO₃, Cu⁺ or Ag⁺ (2).

The olefin purification, with removal of dienes or alkynes, is achieved through a zeolite membrane functionalized with Ag⁺, having a specific catalyst on the permeate side (4), for instance, palladium nanoclusters, in order to promote the hydrogenation reaction of dienes and alkynes to olefins, therefore increasing selectivity, and the separation driving force.

The suitable ceramic membranes for the separation of ethylene from ethane, and for the separation of propylene from propane are silicate based membranes, functionalized with a monolayer of silver nitrate or copper chloride (I).

The most suitable ceramic membranes for the propylene purification, through the removal of propyne and allene, are zeolite based membranes (functionalized with silver or copper, by an ion exchange process), with palladium catalyst nanoparticles at the permeate side. Both selectivity and driving force increase significantly when the propyne or allene (species with higher adsorption affinity and diffusivity) concentration at the permeate side is very low. This can be achieved through the selective hydrogenation of these compounds, promoted by the presence of a catalyst; as soon as these compounds permeate the membrane, reaction takes place on the surface of the catalyst. In order to do so, propane is fed at a pressure between 0.2 and 1.6 MPa and a temperature between 0° C. and 60° C. Simultaneously, hydrogen must be fed (at low pressure) in counter current at the permeate side, at a flowrate slightly above the amount stoichiometrically needed.

### STATE OF THE ART

The separation of olefins from paraffins is one of the most important in the petrochemical industry. The traditional processes: low temperature distillation and extractive distillation, have high-energy requirements and are only attractive for treating streams with high olefins concentration [1, 2].

Up till now, most of the commercially available membranes for gas separations were polymeric. These show a low to medium selectivity and permeability and they only operate under mild conditions [3]. Recent developments in molecular sieve membranes (with pores in the range of nanometers) initiated by Soffer, carbon molecular sieve membranes, and by Barrer and Suzuki, zeolite membranes, [4] show that these membranes have simultaneously high permeabilities and selectivities.

Despite the reduced number of publication on facilitate transport in ceramic membranes, ceramic adsorbents modified with AgNO₃ and CuCl are referred to as showing very high adsorption selectivities towards the olefin/paraffin separations [2].

One of the industrially most important separations is the propylene/propane one [2]. In this separation the olefin (propylene) shows both higher diffusivity and higher adsorption affinity to the membrane. In such cases, there is a synergetic effect, and the bicomponent selectivity becomes higher than the monocomponent one (also known as ideal selectivity) [5]. The most suitable ceramic membranes for this separation are membranes made of the same materials as the corresponding adsorbents, i.e. silicate or carbon molecular sieve functionalized with a monolayer of silver nitrate or copper (I) chloride. These membranes can be supported in ceramic porous materials such as alumina.

A zeolite based membrane functionalized with Ag⁺ or Cu⁺ has a very high adsorption affinity towards allene and propyne [2]. The allene/propylene and propyne/propylene selectivities increase with the decrease of allene and propyne concentrations [2]. Thus, a functionalized ceramic catalytic membrane reactor has a higher performance compared to a simple functionalized ceramic membrane, in terms of selectivity and driving force. The hydrogenation of allene and propyne to propylene has a conversion close to 100%.

Concerning the separation of olefins from the paraffins, ceramic microporous adsorbents functionalized with silver and copper [2], functionalized ion exchange membranes [1] and [2], supported membranes coated with stabilized electrolyte polymers [6] and [7], and liquid gas membrane contactors [1] can be found in the literature. Most of the research was done based on AgNO₃ or AgBF₄ functionalized membranes. Other species were also tested (such as CuCl and PdCl₂) as adsorption and facilitating transport agents (species that can establish π-complexation bounds with olefins). The silver nitrate is considered the best carrier agent for olefin/paraffin separations [2]. Ethylene/ethane selectivity of 2700 and productivity of ethylene of 7.6×10⁻¹⁰ cm³_{NPT} cm⁻² s⁻¹ Pa⁻¹, have been reported when using liquid gas membrane contactors with an ion exchange membrane saturated with silver ions and where the adsorbent is an aqueous solution of silver nitrate [1].

Propylene/propane selectivity above 336 and permeances towards propylene of 1.25×10⁻⁶ cm³_{NPT} cm⁻² s⁻¹ Pa⁻¹, have been reported, when using a silver salts electrolyte stabilized in a polymer membrane [8]. It is the π-complexation bound formed between the silver ion and the olefin that is at the basis of a great sorption selectivity and permselectivity.

Patented processes for olefins/paraffins separation are usually based on: liquid membranes, adsorbents, flat and tubular membranes, aqueous solutions and fractional distillation.

In liquid membranes based processes, aqueous solutions of silver ion complexes are usually employed. U.S. Pat. No. 4,014,665 patent [9] discloses a liquid membrane of this sort where, a small amount of hydrogen peroxide is added for slowing down the reduction of the ionic silver towards metal one. For separating olefins from the paraffins using a liquid membrane, this can be supported using tubular or flat polysulfone membranes [10, 11], or microporous hollow fiber membranes [12].

In the processes where membranes are used to separate olefins from paraffins, cellulose membranes loaded with silver nitrate [13]; carbon molecular sieve membranes [14]; synthetic microporous zeolite membranes [15]; ultra- and micro filtration hydrophobic membranes and non-selective membranes, where a polar solvent is used (e.g. aliphatic polyamine), preferentially arranged in counter-current [16]; selective membranes [17, 18] and polymeric functionalized membranes with silver salts (transport facilitators of olefins) [19 and 20].

An absorption process using aqueous solutions of silver salts, such as silver nitrate can also be employed [21, 22 and 23].

In the adsorbent based processes it can be found type X zeolite adsorbents [24]; type X or Y zeolite adsorbents, with improved olefins adsorption capacities and isomerization and polymerization suppressed catalytic activity [25, 26]; zeolite with a high silica/alumina ratio [27]; or silver based adsorbents [28]. This separation can also be accomplished in a pressure swing adsorption (PSA) unit with activated carbon, silica gel or activated alumina [29] or using silver or copper based adsorbents (forming π-complexation bounds) in PSA units or thermal swing adsorption (TSA) units [30]. The market for olefins separation from paraffins by PSA is increasing especially for small-scale units [2]. The most recent technology for olefins separation from paraffins uses modified adsorbents with silver.

None of the preceding inventions discloses the two key aspects of the present one: the use of microporous ceramic membranes functionalized with an active carrier and; the use of functionalized microporous ceramic membranes containing both an active carrier and a catalyst, where the catalyst is used to increase the separation selectivity and productivity.

### DESCRIPTION OF THE INVENTION

Functionalized microporous ceramic membranes have a higher selectivity when compared to the corresponding functionalized ceramic adsorbents. Indeed, the olefins show higher adsorption affinity and diffusivity when compared to the corresponding paraffins in the functionalized ceramic matrix. As the permeability of a membrane is the product between the adsorption and diffusivity, the ideal selectivity of a membrane is higher than the corresponding adsorption selectivity. Furthermore, it can be experimentally observed that the real selectivity of a membrane (the ratio between the bicomponent permeabilities) is higher than the ideal selectivity (obtained from the ratio between the monocomponent permeabilities), when the permeating species present both higher adsorption affinity and higher diffusivity [5]. Taking this into consideration, it is advantageous to separate the olefins from the paraffins using a membrane process than the corresponding adsorption one.

The ceramic membrane precursors here described are the same as the ones presently used for synthesizing adsorbents for the olefin/paraffin separations. Silicates can be used for synthesizing microporous membrane coated with a silver nitrate monolayer [2]. The membranes should be supported and can be tubular or flat. One of the most convenient supports is the alumina.

Type Y zeolite membranes, exchanged with Ag⁺ or Cu⁺ have to be used for purifying the propylene by removing allene and propyne, and for the purification of ethylene by removing ethyne, once this material shows a high adsorption affinity towards these impurities [2]. As previously mentioned, the performance of these membranes can be largely improved if at the permeate side the impurities concentration becomes very low [2]. This can be attained by selectively hydrogenating the impurities to the corresponding olefins. The present invention also discloses the use of a specific catalyst for hydrogenating the mentioned impurities, such as palladium or platinum nanoclusters and located at the permeate side of the zeolite membrane. In this case, the zeolite membrane works as a catalytic membrane reactor functionalized with a facilitate carrier. The propylene stream containing small amounts of impurities of allene and propyne, for example, should be fed at a pressure between 0.2 and 1.6 MPa to a zeolite membrane functionalized with Ag⁺ or Cu⁺ and containing palladium nanoparticles at the permeate side. To the permeate side an hydrogen stream should be fed in counter-current at a pressure between 5 and 100 kPa, a temperature between 0 and 60° C. and a hydrogen flowrate slightly above the stoichiometric one.

The impurities that selectively permeate the membrane, make contact with the catalyst at the permeate side and, with the presence of hydrogen, they originate the corresponding olefins. The hydrogen should be fed at a flowrate slightly above the stoichiometric value. The permeate pressure should be in the range of hundreds of Pa while the retentate pressure should be the highest possible, ranging between deci-MPa to MPa. This configuration allows a significant reduction in the partial pressure of these impurities at the permeate side, therefore increasing the membrane selectivity [31], and decreasing the pressure driving force necessary for the separation. The fact that, it joins the chemical reaction to a membrane separation unit aiming the increase of the separation selectivity and the decrease of the driving force necessary to the separation is innovative and is one of the key disclosures of the present invention.

### Example 1

### Separation of Olefins from Paraffins

This example illustrates the use of the present invention for the separation of olefins from paraffins originated from an alkylation unit with sulfuric acid as catalyst [13]. The referred stream is fed at a normal flowrate of 100 L min⁻¹ and containing 5% of propane (C₃H₈), 27% of isobutane (C₄H₁₀), 15% of butane (C₄H₁₀), 3% of isopentane (C₅H₁₂), 2.5% of propylene (C₃H₄) and 47.5% of butene (C₄H₄). The olefins permeate the membrane at a normal flowrate of 50 L min⁻¹, containing 5% of propylene and 95% of butene. The paraffins are retained and the retentate flowrate is 50 L min⁻¹, containing 10% of propane, 54% of isobutene, 30% of butane and 6% of isopentane.

### Example 2

### Separation of Olefins from Paraffins with Hydrogenation of Alkynes and Dienes

This example illustrates the use of the present invention on the olefins separation from paraffins and the hydrogenation of the impurities (alkynes and dienes). A typical feed of hydrocarbons [32], containing 1.15% of acetylene (C₂H₂), 71.8% of ethylene (C₂H₄), 13.59% of ethane (C₂H₆), 0.4% of propyne and of allene (C₃H₄), 12.65% of propylene (C₃H₆) and 0.38% of propane (C₃H₈) is fed to the process at a flowrate of 100 mol min⁻¹. The permeate stream is 86.03 mol min⁻¹ and contains 84.83% of ethylene (C₂H₄) and 15.17% of propylene (C₃H₆). The Retentate flowrate is; 13.97 mol min⁻¹, made of 97.28% of ethane (C₂H₆), and 2.72% of propane (C₃H₈).

### DESCRIPTION OF THE FIGURE

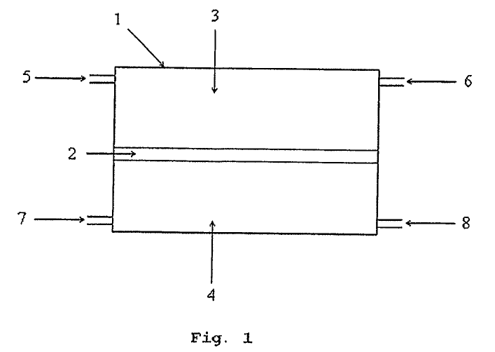

It can be observed in FIG. 1, a divulged device representation of the present invention, which purpose is non-limiting and exemplificative. The referred figure shows:

1. Device for separating the olefins from the paraffins and purification of the paraffins;
2. Membrane;
3. Retentate chamber;
4. Permeate chamber;
5. Feed;
6. Retentate exit;
7. Inlet to the permeate chamber;
8. Permeate exit.

The separation device of olefins from paraffins and olefin purification (1) consists of two chambers, separated by a membrane (2). A mixture of paraffins and olefins (that may contain alkynes or dienes impurities) is fed to the retentate chamber (3) through the feed channel (5). This input is normally made under pressure.

Once in contact with the membrane (2) most of olefins cross it to the permeate chamber (4), where the pressure is lower than in the retentate chamber (3), and exit through the permeate outlet (8). The partial pressure difference of each olefin between the retentate (3) and permeate (4) sides works as the separation driving force. It is then possible to feed to the permeate side (7) a gas stream aiming to dilute the olefin, and then increasing the mentioned driving force and the olefins permeation through the membrane (2).

The paraffins, less permeable through the membrane (2), cross the retentate chamber being withdrawn from the device (1) through the exit channel (6).

If alkynes or dienes impurities are present in the feed stream, these permeate the membrane (2) together with the olefins and are hydrogenated at the catalyst presented at the permeate side (4) of the membrane. The mentioned catalyst converts these impurities in olefins that are collected through the permeate exit (8). For hydrogenation to occur, hydrogen must be fed through the inlet of the permeate chamber (7).

### REFERENCES

[1]—NYMEIJER, K., VISSER, T., ASSEN, R. AND WESSLING, M.; “SUPER SELECTIVE MEMBRANES IN GAS-LIQUID MEMBRANE CONTACTORS FOR OLEFIN/PARAFFIN SEPARATION”; JOURNAL OF MEMBRANE SCIENCE, 232, 107-114, 2004.
[2] —YANG, R. T.; “ABSORBENTS: FUNDAMENTALS AND APPLICATIONS”; JOHN WILLEY, 2003.
[3] —ISMAIL, A. F. AND DAVID, L. I. B.; “A REVIEW ON THE LATEST DEVELOPMENT OF CARBON MEMBRANES FOR GAS SEPARATION”; JOURNAL OF MEMBRANE SCIENCE, 193, 1-18, 2001.
[4] —MENDES, A., MAGALHÃES, F. AND COSTA, C.; “NEW TRENDS ON MEMBRANE SCIENCE”; EDITED BY J. FRAISSARD AND C. W. CONNER IN ‘FLUID TRANSPORT IN NANOPOROUS MATERIALS’, KLUWER, 439-479, 2006.
[5] —LAGORSSE, S., MAGALHÃES, F. D. AND MENDES, A.; “CARBON MOLECULAR SIEVE MEMBRANES—SORPTION, KINETIC AND STRUCTURAL CHARACTERIZATION”; JOURNAL OF MEMBRANE SCIENCE, 241, 275-287 2004.
[6] —KANG, S. W., KIM, J. H., OH, K. S., WON, J., CHAR, K., KIM, H. S. AND KANG, Y. S.; “HIGHLY STABILISED SILVER POLYMER ELECTROLYTES AND THEIR APPLICATION TO FACILITATED OLEFIN TRANSPORT MEMBRANES”; JOURNAL OF MEMBRANE SCIENCE, 236, 163-169, 2004.
[7] —KIM, J. H., PARK, S. M., WON, J. AND KANG, Y. S.; “DEPENDENCE OF FACILITATED OLEFIN TRANSPORT ON THE THICKNESS OF SILVER POLYMER ELECTROLYTE MEMBRANES”; JOURNAL OF MEMBRANE SCIENCE, 236, 209-212, 2004.
[8] —KIM, J. H., WON, J. AND KANG, Y. S.; “SILVER POLYMER ELECTROLYTES BY π-COMPLEXATION OF SILVER IONS WITH POLYMER CONTAINING C═C BOND AND THEIR APPLICATION TO FACILITATED TRANSPORT MEMBRANES”; JOURNAL OF MEMBRANE SCIENCE, 237, 199-202, 2004.
[9] —STEIGELMANN, E. F.; “MEMBRANE PROCESS AND PRODUCT”; U.S. Pat. No. 4,014,665, 1977.
[10] —TSOU, D. T. AND BLACHMAN, M. W.; “FACILITATED LIQUID MEMBRANES FOR OLEFIN/PARAFFIN GAS SEPARATIONS AND RELATED PROCESS”; U.S. Pat. No. 5,135,547, 1992.
[11] —BLACHMAN, M. W. AND TSOU, D. T.; “FACILITATED LIQUID MEMBRANES FOR OLEFIN/PARAFFIN GAS SEPARATIONS AND RELATED PROCESS”; EP0458598, 1991.
[12] —SIRCAR, K. K.; “GAS SEPARATION USING HOLLOW FIBER CONTAINED LIQUID MEMBRANE”; U.S. Pat. No. 6,156,096, 2000.
[13] —KAPLAN, R.; “ISOPARAFFIN—OLEFIN ALKYLATION UTILIZING A MEMBRANE TO SEPARATE OLEFINS FROM A FEED STREAM”; U.S. Pat. No. 4,154,770, 1979.
[14] —AGAM, G., DAGAN, G., GILRON, J., KRAKOV, V. AND TSESIN, N.; “RECOVERY OF OLEFINS FROM GASEOUS MIXTURES”; WO01/17664, 2001.
[15]—HAAG, W. O. AND TSIKOYIANNIS, J. G.; “SEPARATION OF MIXTURE COMPONENTS OVER MEMBRANE COMPOSED OF A PURE MOLECULAR SIEVE”; U.S. Pat. No. 5,069,794, 1991.
[16] —CHEN, T.-J. AND SWEET, J. R.; “OLEFIN/PARAFFIN SEPARATION VIA MEMBRANE EXTRACTION”; U.S. Pat. No. 5,107,058, 1992.
[17] —BAKER, R. W., COSTA, A. R. D. AND DANIELS, R.; “MEMBRANE-AUGMENTED MANUFACTURE OF PROPYLENE DERIVATIVES”; U.S. Pat. No. 6,414,202, 2002.
[18] —ROMAN, I. C., SIMMONS, J. W. AND EKINER, O. M.; “METHOD OF SEPARATING OLEFINS FROM MIXTURES WITH PARAFFINS”; WO2004/050590, 2004.
[19] —KIM, H. S., KANG, Y. S., LEE, B. G., LEE, H. J. AND RYU, J. H.; “SILVER SALT-CONTAINING FACILITATED TRANSPORT MEMBRANE FOR OLEFIN SEPARATION HAVING IMPROVED STABILITY AND METHOD FOR PRODUCING THE SAME”; U.S. Pat. No. 6,706,771, 2004.
[20] —KERRES, J.; “MEMBRANE PRODUCTION FOR OLEFIN/PARAFFIN SEPARATION, COMPRISES USE OF POLYMER MATRIX CONTAINING TRANSITION METAL SALT WHICH REVERSIBLY COMPLEXES UNSATURATED ORGANIC COMPOUNDS AND AVOIDS ADDITION OF WATER OR ALCOHOL TO THE FEED”; DE19929482, 2001.
[21] —MARCINKOWSKY, A. E., KELLER, G. E. AND VERMA, S. K.; “OLEFIN SEPARATION PROCESS”; U.S. Pat. No. 4,174,353, 1979.
[22] —MUNSON, C. L., BOUDREAU, L. C., DRIVER, M. S. AND SCHINSKI, W. L.; “SEPARATION OF OLEFINS FROM PARAFFIN USING IONIC LIQUID SOLUTIONS”; U.S. Pat. No. 6,339,182, 2002.
[23] —PIROVANO, C., SANFILIPPO, D., SAVIANO, F. AND PIOVESAN, L.; “PROCESS FOR THE SEPARATION OF LIGHT OLEFINS FROM PARAFFINS”; U.S. Pat. No. 6,414,210, 2002.
[24] —ROSBACK, D. H. AND NEUZIL, R. W.; “OLEFIN SEPARATION PROCESS”; U.S. Pat. No. 3,969,223, 1976.
[25] —ROSBACK, D. H. AND NEUZIL, R. W.; “OLEFIN SEPARATION PROCESS”; U.S. Pat. No. 4,036,744, 1977.
[26] —ROSBACK, D. H. AND NEUZIL, R. W.; “METHOD FOR MANUFACTURING AND ADSORBENT USEFUL FOR OLEFIN SEPARATION”; U.S. Pat. No. 4,048,111, 1977.
[27] —KANTNER, E., SAVAGE, D. W. AND BELLOWS, R. J.; “ZEOLITE COMPOSITION FOR USE IN OLEFINIC SEPARATIONS”; U.S. Pat. No. 5,292,990, 1994.
[28] —CHO, S. H., HAN, S. S., KIM, J. N., CHOUDARY, N. V., KUMAR, P., GARADI, S. AND BHAT, T.; “ADSORBENTS, METHODS FOR THE PREPARATION AND METHOD FOR THE SEPARATION OF UNSATURATED HYDROCARBONS FOR GAS MIXTURES”; U.S. Pat. No. 6,315,816, 2001.
[29] —MITARITEN, M. J. AND SCOTT, N. H.; “SEPARATION PROCESS FOR THE PRODUCT STREAMS RESULTING FROM THE DEHYDROGENATION OF HYDROCARBONS”; U.S. Pat. No. 5,177,293, 1993.
[30] —YANG, R. T., PADIN, J. AND REGE, S. U.; “SELECTIVE ADSORPTION OF ALKENES USING SUPPORTED METAL COMPOUNDS”; U.S. Pat. No. 6,867,166, 2005.
[31] —PEINEMANN, K. AND OHLROGGE, K.; “SEPARATION OF ORGANIC VAPORS FROM AIR WITH MEMBRANES”; EDITED BY J. CRESPO AND K. BOEDDEKER IN ‘MEMBRANE PROCESSES IN SEPARATION’, NATO ASI SERIES, 272, 357-372, 1993.
[32] —GODÍNEZ, C., CABANES A. L. AND VÍLLORA, G.; “EXPERIMENTAL STUDY OF THE TAIL END SELECTIVE HYDROGENATION OF STEAM CRACKING C₂-C₃ MIXTURE”; CANADIAN JOURNAL OF CHEMICAL ENGINEERING, 74, 84-93, 1996.

1. Catalytic membrane based device capable of purifying olefins from a gas mixture of paraffins, dienes and alkynes, comprising:
a) a feed chamber (3) which allows the admission into the device (1) of the feed gas mixture to be separated and the evacuation of the retentate stream,
b) a permeate chamber (4) that allows the admission of a hydrogen stream (7) and the exit of an olefin concentrated stream,
c) a functionalized microporous membrane (2), which divides the two chambers (3,4) able to accomplish the selective separation of olefins from paraffins, containing, on the side turned towards the permeate chamber, nanoparticles of metal catalyst, capable of promoting a hydrogenation of dienes and alkynes to the corresponding olefins,
wherein the microporous membrane (2) is functionalised through an ionic exchange process, in which the π-complexant active carriers are loaded in the inorganic porous support.

2. Catalytic membrane based device according to claim 1, wherein the nanoparticles of metal catalyst, included in a microporous membrane, are either of nanoparticulated palladium or nanoparticulated platinum.

3. Catalytic membrane based device according to claim 1, wherein the inorganic porous support is a ceramic based material.

4. Catalytic membrane based device according to claim 1, where the inorganic porous support is a zeolite based material.

5. Catalytic membrane based device according to claim 1, wherein the inorganic porous support is a silicate based material.

6. Catalytic membrane based device according to claim 1, wherein the inorganic porous support is a carbon molecular sieve.

7. Catalytic membrane based device according to claim 1 wherein the microporous membrane (2) is functionalized with an active carrier of Ag⁺, Cu⁺ or a mixture of both.

8. Method for purifying olefins from a gas mixture of paraffins, dienes and alkynes, using a catalytic membrane based device of claim 1, wherein the feed chamber is pressurized and the permeate chamber is subjected to low pressure values, comprised between (5 and 30) kPa.

9. Method for purifying olefins from a gas mixture of paraffins, dienes and alkynes, using a catalytic membrane based device, according to claim 8 , wherein the amount of hydrogen fed to the permeate chamber (7) is the same or slightly above 2% of stoichiometric needs.

10. Use of the catalytic membrane based device according to claim 1 wherein it is destined, specifically, to the separation of propylene from propane and of ethylene from ethane.

11. Use of the catalytic membrane based device according to claim 1 wherein it is destined to the purification of olefins with the removal of dienes and alkynes.
